(19) United States
(12) Patent Application Publication
(10) Pub. No.: US 2008/0210238 A1
(43) Pub. Date: Sep. 4, 2008

(54) Manual resuscitators

(75) Inventor: Mark James Huddlestone, St. Ouen (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 12/073,110

(22) Filed: Feb. 29, 2008

(30) Foreign Application Priority Data
Mar. 3, 2007 (GB) 0704138.7

(51) Int. Cl. A61M 16/00

(52) U.S. Cl. 128/205.14; 128/205.13

(57) ABSTRACT

A manual squeeze bag resuscitator has a strap assembly extending around its central portion. The length of the strap assembly is adjustable so that it can limit the diameter of the bag and hence limit its effective volume, such as for use with smaller patients.

### BACKGROUND OF THE INVENTION

This invention relates to manual resuscitators.

The invention is more particularly concerned with manual resuscitator bags of the kind that are squeezed manually to deliver ventilation gas to a patient.

Manual squeeze bag resuscitators are commonly used to ventilate a patient, particularly in emergency situations where a mechanical ventilator is not available. The bag has an inlet at one end with a valve that allows air to flow into the bag but prevents air escaping from the inlet when the bag is squeezed. The bag outlet is normally provided with a patient valve that allows gas from the bag to flow to the patient and diverts exhaled gas from the patient through an exhaust port. When the bag is squeezed, air flows from the bag via the patient valve to inflate the lungs of the patient. The bag is then released so that its resilience allows it to recover its original shape and so that air fills the bag through the inlet. Although these resuscitators have been used widely for many years they do suffer from various disadvantages. First, it is difficult accurately to alter the volume delivered for different patients. In emergency situations, such as in ambulances, it is usual only to carry a resuscitator bag of a standard size. The resuscitators are usually made of a size sufficient to suit an adult man so, when the resuscitator needs to be used on a child or someone of slight build, the user has to estimate the extent to which the bag should be squeezed in order not to over-inflate or under-inflate the patient. A second disadvantage is that prolonged use of a squeeze bag resuscitator can be tiring on the hands of the user.

### BRIEF SUMMARY OF THE PRESENT INVENTION

It is an object of the present invention to provide an alternative manual resuscitator.

According to one aspect of the present invention there is provided a manual resuscitator having a resilient squeeze bag, an inlet at one end of the bag that allows gas to flow into the bag and an outlet at the opposite end of the bag by which gas flows to the patient, the resuscitator including adjustable restrictor means arranged to limit the effective volume of the bag.

The restrictor means is preferably elongate and flexible along its length. The bag preferably has a portion of circular section, the restrictor means extending around the portion of circular section. The restrictor means may include a member extending on the outside of the bag and adjustable to limit the diameter of the bag. The restrictor means preferably includes a strap assembly extending around the bag. Opposite end portions of the strap assembly may have cooperating hook and loop formations so that they can be pressed into engaging contact with one another. The strap assembly may include two parallel straps arranged to extend circumferentially around the bag and two lateral webs joined to both straps at opposite ends. The restrictor means may be marked to indicate where it should be set for patients of different builds.

According to another aspect of the present invention there is provided restrictor means for a resuscitator according to the above one aspect of the present invention.

A resuscitator according to the present invention will now be described, by way of example, with reference to the accompanying drawing.

### BRIEF DESCRIPTION OF THE DRAWING

FIG. 1 is a perspective view of the resuscitator from one side; and

FIG. 2 is a plan view of the strap assembly unwound from the resuscitator.

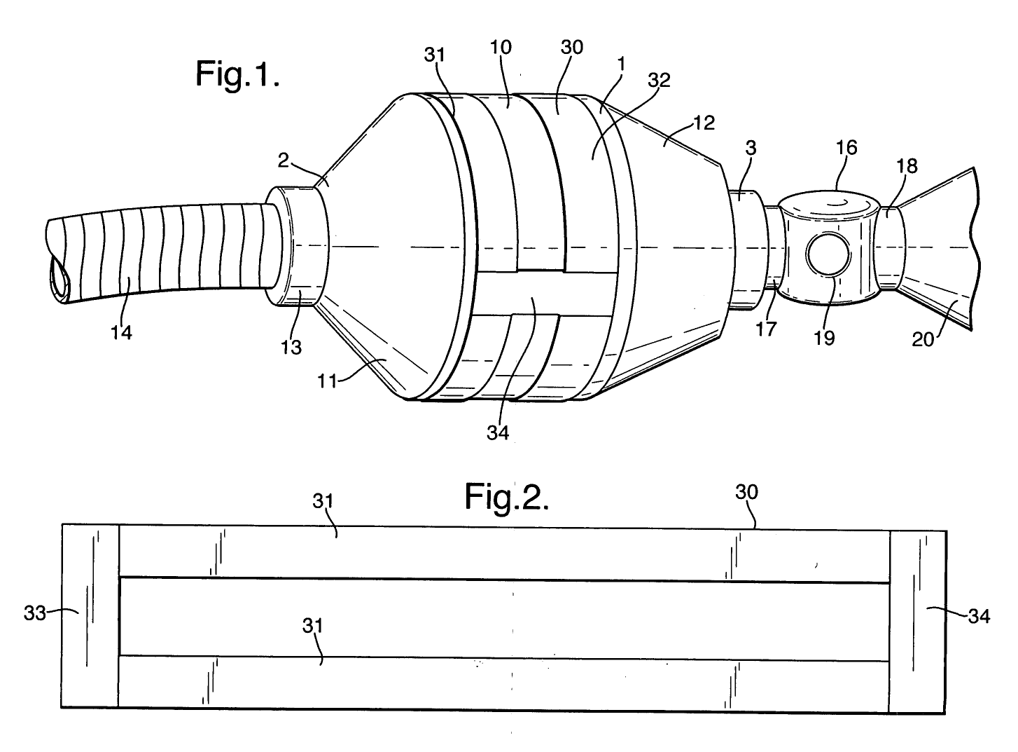

### DETAILED DESCRIPTION OF THE PREFERRED EMBODIMENT

The resuscitator takes the form of a resilient bag 1 with an inlet 2 at its left-hand end and an outlet 3 at its opposite end.

The bag 1 is made of a resilient plastics material and has a central cylindrical portion 10 of circular section and opposite tapering portions 11 and 12 at the inlet and outlet ends respectively. A rigid valve assembly 13 is bonded into the inlet end 2 of the bag, the valve assembly being arranged to allow gas to flow into the bag from outside and to prevent gas flow in the opposite direction. It will be appreciated, however, that the valve assembly 13 need not totally block flow from the bag. The valve assembly 13 is attached to a corrugated flexible hose 14 about 1 m long, the other end of which is open to atmosphere. A small bore oxygen tube (not shown) may extend along the hose 14 and be attached at one end to the valve assembly 13 so as to enable supplementary oxygen to be supplied to the resuscitator if needed. Instead of being closed by a valve, the inlet could open into an open-ended tube of fixed or variable length.

At its outlet end 3 the bag 1 is bonded to a rigid patient valve assembly 16 of conventional construction. The valve assembly 16 has an inlet 17, a patient outlet 18 and an exhaust port 19. The valve assembly 16 includes a movable valve element (not shown), which operates in the usual way, to allow gas to flow from the bag to the patient outlet 18 and to divert gas exhaled by the patient to the exhaust port 19. The patient outlet 18 is connected to a face mask 20. Alternatively, the patient outlet 18 could be connected to a breathing tube such as a tracheal tube.

As so far described, the resuscitator is conventional.

The resuscitator differs from previous resuscitators in that it has an adjustable restrictor by which the effective volume of the bag can be limited. The restrictor takes the form of a strap assembly 30 embracing the central, cylindrical portion 10 of the bag. The strap assembly 30 comprises two narrow parallel straps 31 and 32 spaced from one another by a distance about equal to the width of the straps. The straps 31 and 32 are joined with one another at opposite ends by lateral webs 33 and 34 extending at right angles to the straps. The straps 31 and 32 have cooperating hook and loop formations on opposite surfaces, in particular, the outwardly-facing side of the straps is formed with loops whereas the inwardly-facing side is formed with hooks. The length of the strap assembly 30 exceeds the circumference of the central portion 10 and is typically about 1.5 times the circumference so that the ends of the strap assembly overlap one another by about one third of the length of the assembly. The hook and loop formations on the overlapping parts of the strap assembly 30 engage one another and provide a high shear strength connection. The strap assembly 30 is held on the outside of the bag 1 by friction and the compressive force exerted by the strap assembly. Alternatively, the strap assembly could be permanently secured to the bag, along most of its length or just towards one end, such as by an adhesive, fasteners or welds. Alternatively, parts of the strap assembly could extend through loops or eyes moulded with the bag. It will be appreciated that the strap assembly 30 is substantially inelastic or non-extensible such that it does not allow the compressed bag to expand beyond the diameter set by the strap.

In normal use, on an adult of normal or large build, the strap assembly 30 is fastened relatively loosely so as not to compress the wall of the bag to any significant extent. The user can then use the bag in the normal way by gripping and squeezing the central portion 10 on top of the strap assembly 30. The flexible nature of the strap assembly does not impeded compression of the bag.

If the resuscitator is to be used with a child or person of small build, the user assesses the lung capacity of the patient, unpeels the outer end of the strap assembly, tightens the strap assembly 30 and reapplies the outer end 34 to the strap at a location further around the strap so as to reduce its effective circumference. The strap assembly may be marked with appropriate legends to indicate where it should be fastened when used with patients of different builds. With the strap assembly 30 in this setting, the cylindrical central portion 10 is compressed to a smaller diameter than its natural diameter. The effective internal volume of the bag 1 is, therefore, reduced. When the user squeezes the bag 1, it will be appreciated that the volume of gas discharged to the patient will be correspondingly reduced.

The arrangement of the present invention, therefore, enables the stroke volume of the resuscitator to be optimized so that the patient is not over-inflated, which can cause damage and hinder recovery. Where conventional bags are used on smaller patients, there is a risk that the user will overcompensate and inadequately squeeze the bag for fear of over-inflating. This can leading to insufficient ventilation. By controlling the maximum volume the user is able to use, the user is given greater confidence in safe use of the resuscitator, thereby reducing the risk that he will inadequately ventilate smaller patients. When the volume of the resuscitator is reduced for smaller patients, the user needs to apply less force to achieve correct ventilation. This can help reduce fatigue over prolonged use. The smaller external dimension of the bag when compressed also makes it easier to use by someone with small hands.

Various modifications to the invention are possible.

The strap could be resilient, being made, for example, of an elastomeric material, such as neoprene or natural rubber. Instead of using hook and loop fastening material to retain the length of the strap, a ratchet arrangement could be used similar to those on cable ties. A part of the length of the strap would be ribbed and it would be shortened by pulling one end where it extends through a ratchet at the opposite end of the strap and arranged to catch on the ribs. Alternatively, a screw mechanism could be used to shorten or lengthen a strap or similar elongate member encompassing the bag; the screw mechanism could be graduated to show the volume of the bag. In another arrangement, the strap or the like could resemble a tourniquet, with opposite ends of the strap being twisted together to shorten its length. A draw-string arrangement could be used. The strap need not be flexible along its entire length. Instead, it could have rigid sections that indent into the bag and alter its volume. The bag need not be circular in section but could have other shapes. For example, the bag could have one or more folded tucks in its surface or the centre portion could be fluted longitudinally or laterally so that the flutes concertina closed when compressed, thereby reducing the volume.

What I claim is:

1. A manual resuscitator comprising: a resilient squeeze bag having a volume; an inlet at one end of the bag that allows gas to flow into the bag; an outlet at an opposite end of the bag by which gas flows to a patient; and a restrictor arranged to limit the effective volume of the bag.

2. A resuscitator according to claim 1, wherein the restrictor is elongate and flexible along its length.

3. A resuscitator according to claim 1, wherein the bag has a portion of circular section and wherein the restrictor extends around said portion of circular section.

4. A resuscitator according to claim 3, wherein the restrictor includes a member extending on an outside of the bag and adjustable to limit the diameter of the bag.

5. A resuscitator according to claim 1, wherein the restrictor includes a strap assembly extending around the bag.

6. A resuscitator according to claim 5, wherein opposite ends of said strap assembly have hook and loop formations so that they can be pressed into engaging contact with one another.

7. A resuscitator according to claim 5, wherein said strap assembly includes two parallel straps arranged to extend circumferentially around the bag and two lateral webs joined to both straps at opposite ends.

8. A resuscitator according to claim 1, wherein the restrictor is marked to indicate where it should be set for patients of different builds.

9. A resuscitator according to claim 1, wherein said inlet includes a one-way valve arranged to allow gas to enter the bag but to prevent gas flowing out of the bag through said inlet.

10. A resuscitator according to claim 1, wherein said outlet includes a patient valve arranged to allow gas to flow out of the bag to a patient and to divert gas exhaled by the patient to atmosphere.

11. A manual resuscitator comprising: a resilient squeeze bag having a volume; a one-way valve at one end of the bag arranged to allow gas to flow into the bag and to prevent gas flowing out of the bag; a patient valve at an opposite end of the bag by which gas flows to a patient and gas exhaled by the patient is diverted to atmosphere; and a flexible strap assembly extending around the outside of the bag that is selectively adjustable to vary the effective volume of the bag.

12. A method of ventilating a patient, including the steps of: providing a squeeze bag resuscitator of the kind having a volume, an inlet at one end of the bag that allows gas to flow into the bag, an outlet at an opposite end of the bag by which gas flows to a patient, and a restrictor arranged to limit the effective volume of the bag; assessing the lung capacity of the patient; adjusting the restrictor to alter the volume of the bag to suit the patient; and then squeezing the bag repeatedly to ventilate the patient.
